(19) United States
(12) Patent Application Publication
(10) Pub. No.: US 2021/0033810 A1
(43) Pub. Date: Feb. 4, 2021

(54) FIBER OPTIC CABLE LIGHT SHIELD

(71) Applicant: John Temple, Chelsea, MI (US)

(72) Inventor: John Temple, Chelsea, MI (US)

(21) Appl. No.: 16/524,499

(22) Filed: Jul. 29, 2019

(51) Int. Cl. G02B 6/44

(52) U.S. Cl. CPC G02B 6/4436; A61B 1/00126

(57) ABSTRACT

An improved shield is adapted for use on a light cable that attaches to a surgical viewing instrument such as a laparoscope. When the cable becomes detached, the shield closes off the light-emitting end of the cable, substantially preventing light from escaping, where the heat may start a fire or burn a patient. A hollow, tubular body is composed of a flexible, resilient, temperature-resistant material such as a high-temperature silicone having proximal and distal ends defining a length. The distal end includes only a plurality of slits extending radially outwardly from a center point. The slits define fine gaps that do not allow light from the cable to escape when detached. The tubular body may be integrally molded and light-translucent with a cylindrical or frustoconical shape. The shield may be perforated, but only in areas that do not allow light to escape when the light cable becomes detached.

### FIELD OF THE INVENTION

This invention relates generally to minimally invasive surgical procedures and, in particular, to a light shield configured for use with optical cables used in laparoscopic procedures.

### BACKGROUND OF THE INVENTION

Laparoscopy is a minimally invasive surgical diagnostic procedure used to examine the organs inside the abdomen. At least one small incision is made into the abdominal space, which is inflated with CO₂ to better visualize internal organs. One incision holds a trocar through which a laparoscope is inserted. The laparoscope typically transmits images to a monitor for viewing. Other incisions may be made for other instruments to remove tissue, perform biopsies, and so forth.

Modern laparoscopes are long, thin tubes with integrated high-resolution cameras. High-intensity light from an optical fiber cable is coupled to the side of the instrument near the proximal end outside the body. The light is carried to the tube to the distal end where it illuminated the field of view.

The fiber-optic cable typically connects to the laparoscope through a threaded connection, allowing the cable to be exchanged or removed for maintenance or cleaning. On occasion, due to frequent manipulation of the instrument by the surgeon, this threaded connection can become detached, allowing the tip of the fiber cable to fall freely onto the patient, drapery, or other surrounding surfaces. This exposed tip is very hot, and can result in burning, even fires, if left unchecked.

There are a couple of existing devices designed to address this problem. One solution is described in published international WO2017152266A1, entitled “The Light Cable Safety Sleeve.” The device described in this reference is a single use, silicone sleeve with perforations on the shaft for heat release/dissipation, “and shaped collars at each end for placement stability and suspension of the light cable and connection assembly (within the sleeve).” One issue with this device is that it is essentially open at both ends, such that considerable light escapes through the distal end and does not adequately solve issues with overheating.

A commercially available device is the Jackson Medical Light/Heat Shield called the GloShield™. However, this device comes in different sizes, requiring a user to purchase and use a model that fits a particular light cable. This device is also awkward to use in the field, requiring exact positioning on the cable, which can be slide when attaching it to the scope leaving it out of position.

There is an outstanding need, therefore, for an optical cable light shield that fits different products while effectively blocking the light and heat should the cable become disconnected from the side of the viewing instrument.

### SUMMARY OF THE INVENTION

This invention resides in an improved shield for a light cable. The device may be used on a light cable that attaches to a surgical viewing instrument such as a laparoscope, such that when the cable becomes detached, the shield closes off the distal end of the cable, substantially preventing light from the cable to escape, where the heat may start a fire or burn a patient.

The improved light shield comprises a hollow, tubular body composed of a flexible, resilient, temperature-resistant material such as a high-temperature silicone. The hollow, tubular body has proximal and distal ends defining a length. The proximal end includes a central aperture and a plurality of slits extending radially outwardly from the central aperture forming a plurality of flexible leaves or flaps. However, in contrast to existing devices, the distal end includes no apertures, but instead includes only a plurality of slits extending radially outwardly from a center point.

When a fiber optic cable is threaded through the improved light shield, the material between the slits form flexible flaps that the cable extends through. If the cable becomes detached, however, the flaps close, leaving only the slits, which define fine gaps that do not allow light from the cable to escape.

In the preferred embodiment, the hollow, tubular body is an integrally molded component that may be light-translucent. The length of the body may be in the range of 60 to 100 mm with a cylindrical or frustoconical shape, wherein the proximal end of the device has a diameter that is larger than the diameter at the distal end. The shield may further include a plurality of perforations along the length of the body, but only in the proximal half of the body to further ensure that light does not escaped if and when the fiber-optic light cable becomes detached.

### BRIEF DESCRIPTION OF THE DRAWINGS

FIG. 1 is side view of a preferred embodiment of the invention;

FIG. 2 is a front view of the distal tip of the preferred embodiment;

FIG. 3 is a back view of the proximal end of the preferred embodiment;

FIG. 4 is a cross section of the device of FIGS. 1-3; and

FIG. 5 is an oblique view of the device on a fiber cable that has become disconnected from a laparoscope, illustrating the way in which the distal tip closes to block the light from the cable.

### DETAILED DESCRIPTION OF THE PREFERRED EMBODIMENTS

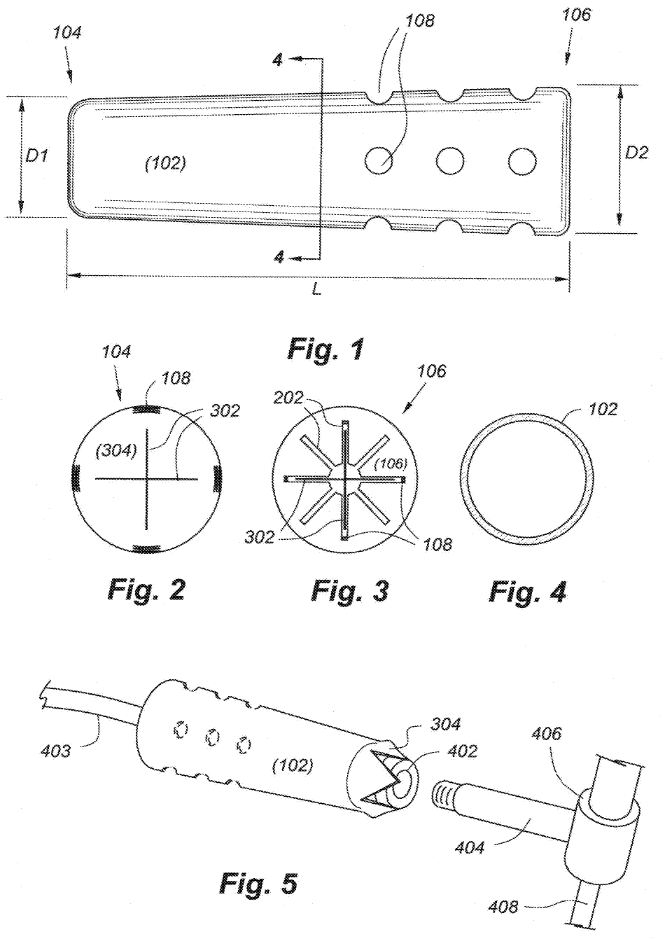

Now making reference to the accompanying drawings, FIG. 1 is side view of a preferred embodiment of the invention. The device broadly comprises a unitary article composed of a temperature-resistant flexible yet resilient material. In the preferred embodiment, the material is a high-temperature silicone manufactured by WYNCA TINYO SILICONE CO., LTD,” their product number TY971-60, and is formed using a silicone compression molding process.

The device has an overall length in the range of 60 to 100 mm, more preferably in the range of 80 to 90 mm, and most preferably about 84 mm. As can be seen, the device tapers somewhat from the proximal end 106 to the distal end 104. The diameter of the proximal end, D2, may be in the range of 20 to 35 mm, more preferably 20 to 25 mm, and most preferably about 25 mm, while the diameter D1 of the distal end may be in the range of 15 to 35 mm, more preferably in the range of 15 to 25 mm, and most preferably about 20 mm.

The preferred embodiment includes a plurality of perforations 108, which may be of any shape including circular with diameters in the range of 2 to 10 mm, more or less. For example, circular perforations with diameters of about 4.5 mm may be used. While these perforations are provided for enhance air circulation, they may be eliminated. In all embodiments, however, there are no perforations of any kind on the distal half of the device, that is, from L/2 to the distal tip, including the tip itself as discussed below.

FIG. 2 is a view of the proximal end of the device. The preferred embodiment uses a star-shaped opening including a central aperture 204 with spoke-like slots 202. The central aperture 204 may have a diameter in the range of 0 to 5 mm or more; however, in the preferred embodiment the central aperture is about 5-6 mm, more preferably 5.5 to accept a wide range of commercially available light cables. The spokes 202 may have gaps in the range of 1 mm, more or less. Note that while eight leaves and gaps are shown, the number is not critical in that more or fewer may be used. Even a single gap forming two leaves may be used; however, such a design may impede sliding on the fiber optic cable.

FIG. 3 is an end view of the distal tip 104. In contrast to existing designs, the tip includes multiple leaves 304, but with no central aperture and wherein the separations between the leaves are cuts or slices leaving no discernable gaps. As such, when the leaves are closed, the distal end is effectively closed off, allowing little—if any—direct light from the fiber optic able to penetrate into the surrounding environment. That is, while the device may glow due to the translucent nature of the silicone material, virtually no direct light rays will penetrate through the device to cause overheating of the patient or surrounding surfaces. In the preferred embodiments, the width of the slits 302 is 0.01 mm or less.

In FIG. 3, while a fixed number of slits 302 and leaves are shown (i.e., four), this number may also vary from 2 to 6 or more. However, it was discovered that four leaves and slits provides an appropriate force to drawn the loose tip of the light cable back into the device so that it closes off.

FIG. 4 is a cross section of the device, and FIG. 5 is an oblique view of the device on a fiber cable that has become disconnected from a laparoscope, illustrating the way in which the distal tip closes to block the light from the cable. FIG. 5 shows the device 402 on a light cable 403, and wherein the connector 402 has become disengaged from the corresponding connector 404 on the laparoscope 406. This Figure also shows the viewing tube 404 that is inserted into a body cavity.

In FIG. 5, the flaps of end 304 have separated to accommodate connector portion 402. However, due to the thickness and resilient nature of the material, this deformity creates a force causing the cable tip 402 to slip back into the device, with cable 403 sliding slightly out through the proximal end 106, such that the light-emitting tip of the cable is fully encased within the body of the device 102 as leaves 304 close. Again, while the distal end of the device will glow, due to the lack of apertures on the front half of the unit, no direct light will escape.

1. A shield for a light cable, comprising:
a hollow, tubular body composed of a flexible, resilient, temperature-resistant material;
the hollow, tubular body having proximal and distal ends defining a length;
wherein the proximal end of the hollow, tubular body includes a central aperture and a plurality of slits extending radially outwardly from the central aperture forming a plurality of flexible leaves; and
wherein the distal end of the hollow, tubular body includes no apertures, but instead includes a plurality of slits extending radially outwardly from a center point.

2. The shield of claim 1, wherein the hollow, tubular body is an integrally molded component.

3. The shield of claim 1, wherein the hollow, tubular body is composed of temperature-resistant silicone.

4. The shield of claim 1, wherein the flexible, resilient, temperature-resistant material is translucent.

5. The shield of claim 1, wherein the length of the hollow, tubular body is in the range of 60 to 100 mm.

6. The shield of claim 1, wherein the length of the hollow, tubular body is cylindrical in shape.

7. The shield of claim 1, wherein the length of the hollow, tubular body is frustoconical in shape, and wherein the proximal end of the body has a diameter that is larger than the diameter of the distal end.

8. The shield of claim 1, wherein the slits extending radially outwardly from the central aperture in the proximal end of the body form gaps with spaced-apart edges.

9. The shield of claim 8, wherein the edges of the gaps are spaced apart by about 1 mm.

10. The shield of claim 1, wherein the central aperture in the proximal end of the body is a circle with a diameter in the range of 5 to 6 mm.

11. The shield of claim 1, wherein the slits in the distal end of the body have a width of 0.01 mm or less.

12. The shield of claim 1, wherein the proximal end of the body has 8 slits and leaves.

13. The shield of claim 1, wherein the distal end of the body has 4 slits and leaves.

14. The shield of claim 1, further including a plurality of perforations along the length of the body, but only in the proximal half of the body.
